(19) United States
(12) Patent Application Publication
(10) Pub. No.: US 2022/0401700 A1
(43) Pub. Date: Dec. 22, 2022

(54) DEVICE FOR SECURING A CATHETER

(71) Applicants: Brian SHAY, Edgewood, KY (US); Michael SHAY, Pittston, PA (US)

(72) Inventors: Brian SHAY, Edgewood, KY (US); Michael SHAY, Pittston, PA (US)

(21) Appl. No.: 17/775,692

(22) PCT Filed: Nov. 23, 2020
(86) PCT No.: PCT/US2020/061758

§ 371 Date: May 10, 2022

Related U.S. Application Data
(60) Provisional application No. 62/939,840, filed on Nov. 25, 2019.

(51) Int. Cl. A61M 25/02

(52) U.S. Cl. CPC A61M 25/02; A61M 2025/0266; A61M 2025/028

(57) ABSTRACT

A device for securing a catheter inserted into an incision or orifice of a patient is described. The device includes a module with bore into which the catheter can be inserted. Semirigid tethers connected to the module and to the patient prevent movement of the catheter relative to its position in the incision or orifice.

### CROSS-REFERENCE TO RELATED APPLICATION

This application claims the benefit of priority to U.S. Provisional Patent Application Ser. No. 62/939,840 filed Nov. 25, 2019, the contents of which are incorporated herein in their entirety.

### BACKGROUND

Catheters and their use are necessary parts of patients' medical treatment be it surgery, infusion of pharmaceuticals or biologicals or promoting removal of bodily fluids. The typical tubular shaped, flexible catheter can be inserted, implanted and generally placed into and through a patient's orifices and ducts, can be inserted into arteries, veins or lymphatic vessels and can be surgically installed through and into the patient's abdominal and chest (heart, lung, stomach, gut) spaces. A typical catheter of this kind is a Foley catheter which is passed through the urethra to the bladder to enable removal of urine. The implanted and/or inserted catheter often dwells in the patient longer than a few minutes. Although the comfort factor is not an issue during surgical procedures because the patient is usually sedated and/or has been given a local anesthetic, the post procedure recovery differs. Often the patient recognizes and reacts to the catheter's uncomfortable and sometimes irritating presence. Medical personnel caution the patient not to pull, move or otherwise disturb the catheter but the irritation factor often wins out. The patient tugs, pulls and/or attempts to adjust the catheter position to relieve the irritation and pain. This kind of patient irritation, pain and activity often occurs with a Foley catheter. This action, however, can lead to untoward results. A dislodged catheter can damage a vessel wall or harm an internal organ. Dislodgement can interrupt the function of the catheter leading to buildup of body fluids, toxins and other undesirable results.

Appropriate nursing techniques foresee such problems and are routinely practiced with some success to minimize the irritation factor in patient settings. Appropriate nursing instruction to deal with irritation factors in a home environment are also routine. Nevertheless, the “urge to pull” exists even when balms and other calming, soothing factors, medicaments and tapes are applied.

Therefore, it is a goal of the present invention to develop an adjunct to good catheter care so that the “urge to pull” and its execution will not cause dislodgement of the implanted or inserted catheter. A further goal is the development of devices and techniques to hold the catheter in place when the “urge to pull” occurs.

### SUMMARY

Embodiments of the present invention are directed to a device for securing a catheter when implanted or inserted onto a patient. Embodiments of the device include a module adapted to fit around and/or be combined with the catheter and tethers with distal ends removably attached to the module and proximal ends for removable attachment to the patient through use of an adherent material.

Embodiments of the device may be employed with catheters inserted into veins, arteries or lymphatic vessels or body organs for temporary or long term use and/or implanted into the body for periodic and permanent or semi-permanent delivery of medicaments. Embodiments of the device may be employed with Foley catheters, mammary duct catheters, urethra catheters, anal catheters, intravenous catheters, arterial catheters, nasogastric tubes, percutaneous endoscopic tubes, gastrostomic tubes and catheters and/or medical tubes and/or cannulas for insertion into living organisms and cannulas and/or catheters for attachment to implanted reservoirs such as insulin reservoirs. Especially preferred is the device for use with Foley catheters inserted into men and women.

Embodiments of the module component may comprise a solid construct having a three-dimensional shape of a sphere, ellipsoid (egg shape), cube, block, or other polygonal shape especially with round corners and rounded edges. The module has an outer surface, a front, a back, a center axis traversing the module from the front to back and a cross sectional mid portion incorporating the center axis. The module further comprises a bore traversing the midportion of module from the front to the back of the module along the center axis.

A catheter may be inserted into the bore of the module or may be preinstalled in the bore so as to form a conjoined module and catheter. The module bore may be configured to provide a snug fit and/or immovable fit around the catheter.

One embodiment of the module component may comprise a single, unitary body construct containing the bore. This module embodiment as a single unitary body combines with the catheter by insertion of the catheter into the module bore. The module position on the catheter may arranged so as to provide an appropriate length of catheter for use with the patient. Alternatively, the module may be installed on the catheter after the catheter is inserted into the patient. In this unitary body embodiment of the module, the bore wall may be configured to bind mechanically and/or frictionally to the outer surface of the catheter and/or may contain an adhesive coating to adhere to the outer surface of the catheter. Alternatively, this embodiment of the module may include one or more snap or clamp fittings surrounding the bore opening at the back and/or front of the module which may be closed around the catheter to hold the catheter in place.

Another embodiment of the module component may comprise a sectioned construct having at least section halves with the section division plane or planes preferably including the cross sectional mid portion and the bore. The sections may fit together by one or more snap fits on the mating section surfaces, by adhesive of the mating section surfaces or by binders on the outer surfaces for holding the sections together. In this section embodiment of the module, the bore wall may be configured to bind mechanically and/or frictionally to the outer surface of the catheter and/or may contain an adhesive coating to adhere to the outer surface of the catheter.

A further embodiment of the module component may comprise a combined unitary construct of the module permanently formed onto the catheter.

The module and tethers may be constructed of any medically acceptable substance including but not limited to a plastic, a rubber material (cross linked polyolefin, preferably elastomeric), a silicone material, a metal and/or gel such as a shape retaining gel and/or any combination thereof. Preferably, the substance may be capable of being molded, extruded, milled, melt formed, or other appropriate process to form the medical device. Plastics may be thermoplastic or thermoset and may be polyolefins, poly(meth)acrylates, polyesters, polyamides, polycarbonates, polyurethanes, polyureas or any combination thereof.

At least two tethers having proximal and distal ends and a rod, slat, plank, band, ribbon or strip shape that is rigid to semiflexible enable connection of the module to the patient. The distal ends may be removably or permanently joined to the outer surface of the module and the proximal ends may be removably attached to an adherent material. The adherent material such as but not limited to adhesive bandage material enables attachment to the patient's skin. An exemplary use of this arrangement is insertion of a Foley catheter through the urethra and into the bladder of male and female patients.

Alternatively, when device embodiments are to be employed with a Foley catheter for use with male patients, the proximal ends of the tethers may be removably joined with an adjustable ring to be fitted around the patient's male organ. The ring may be made of the same medically acceptable substance as the module and includes components to render the ring flexible. The ring may be constructed to have a split through the wall of the ring and have a snap fit arrangement on opposing spit surfaces for closing the split together. The flexibility of the ring combined with the split and snap fit enables the ring to be tightened to a variable diameter. The snap fit is designed to hold the ring in an adjustable but fixed diameter when the snap fit is closed. The inner surface of the ring may include a tacky material to enable the ring to temporarily affix the ring to the skin of the male member (penis). After fitting the module and tethers to the inserted Foley catheter, the adjustable ring in an open condition may be fitted to the proximal ends of the tethers and around the male member. The ring may be closed and the snap fit connected to provide a non-slip fit of the ring around the male member. The adjustable diameter of the ring is designed to fit all sizes of male members.

### BRIEF DESCRIPTION OF THE FIGURES

FIG. 1 depicts a unitary module with bore entrance clamp and catheter.

FIG. 2 depicts a sectioned module with tethers and catheter.

FIG. 3 depicts a unitary module with permanently attached catheter.

FIG. 4 depicts a module and Foley catheter in use in a male patient.

FIGS. 5A and 5B depict a split ring with snap fit for use with a Foley catheter.

### DETAILED DESCRIPTION

The invention is directed to embodiments for securing a catheter and substantially to essentially preventing its movement relative to its implantation or insertion site. The embodiments of the invention enable securing the catheter to the surrounding skin of the patient through a module component surrounding and secured to the external portion of the catheter and secured through tethers to the patient's skin. A preferred version of these embodiments of the invention is the combination of the module and tethers for use with a Foley catheter for female and male patients.

### Definitions

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers.

“Substantially” and “substantial” as the terms are used herein mean completely or almost completely; for example, a binding fit that is “substantial” means that the fit together of two components is such that they cannot be pulled apart by reasonable force applied by a human hand.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50 percent relative humidity. All such weights as they pertain to listed features are based on the application level unless otherwise specified.

“Kit,” as used herein, means a packaging unit comprising a plurality of components. An example of a kit is, for example, a module, tethers and catheter for insertion into the module. Another kit may comprise a module and tethers for use with a separately packaged catheter. A kit may also comprise assembly and application instructions demonstrating how to insert and lock the catheter in the module and how to measure and/or cut appropriate lengths for the tethers.

“Catheter” as used herein means a tube of any construction, size, length suitable for insertion into any orifice, duct and/or through skin into an internal organ and/or tissue and/or vessel of a living organism such as a mammal and especially a human. The tube may be flexible or semi-rigid, preferably flexible and of a size appropriate for the orifice, duct and the like as described above for the kind of mammal in need of the catheter. Typical material forming a catheter is a synthetic or natural polymer, rubber (cross linked polyolefin), natural or synthetic latex, silicone polymer, organic polymer and similar materials.

“Snap fit” is a pair of parts with one having a flanged projection that fits into another having a receptacle for the projection. When pushed or snapped together, the snap fit parts firmly bind together but can be pulled apart with force. The snap fit may be employed to bind the tethers to the module and/or to hold sections of the module together.

Velcro® connection is a pair of hook and eye strips designed to hold two pieces together tightly. The Velcro® connection may be employed to bind the tethers to the module and/or to hold sections of the module together.

### Embodiments of the Device

As described in the Summary, the Device for securing a catheter according to the invention comprises at least a module, tethers and optional adhesive material. The module may be installed onto the catheter following insertion of the catheter into the patient or alternatively the module may be installed onto the catheter prior to catheter insertion into the patient. The former procedure calling for installation of the module post catheter insertion into the patient is preferred at least to efficiently preserve sterile conditions of the catheter. Alternatively, the catheter and module may be permanently conjoined when manufactured. Embodiments of the module include: a) a unitary body with bore, b) a sectioned module with bore in which the sections intersect the bore, and c) a permanently conjoined module and catheter. An example of the device in especially preferred use includes but is not limited to the device with a Foley catheter for male and female patients. Details of each are described.

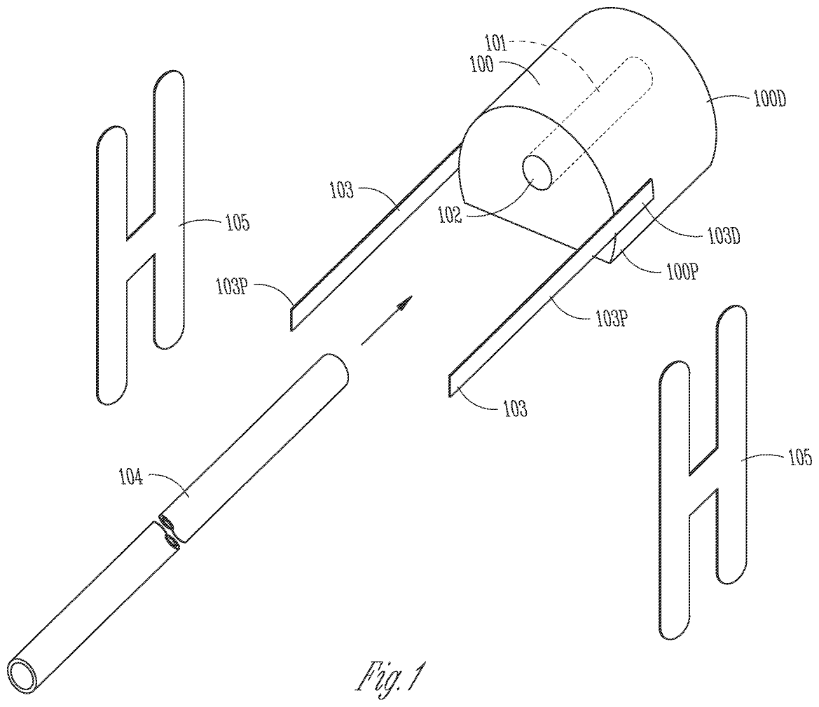

Embodiments of the unitary body module and tethers according to the invention (FIG. 1) have dimensions suitable for use with any kind of catheter. Examples of such catheters include but are not limited to intravenous and/or arterial catheters inserted in the arm or leg, male and female Foley catheters, vaginal catheters, and any form of catheters for insertion into living organisms such as but not limited to humans, cannulas functioning as catheters to be inserted into living organisms and cannulas/catheters for connection to an indwelling portal functioning as a reservoir for a pharmaceutical agent. An example of such a reservoir is an indwelling insulin reservoir and pump. The overall dimension of the unitary body module is configured so as to provide a comfortable, non-bulky arrangement on the patient's body.

FIG. 1 illustrates an embodiment directed to the unitary body module 100 with insertable catheter 104. Module 100 has bore 101 traversing the long axis of module 100. The bore is positioned approximately midpoint cross section in module 100. Tethers 103 are attached to module 100 at their distal ends 103d by a binding adhesive, by Velcro® connection of the tethers and module or by a snap fit arrangement (not shown) of a snap projection and snap receptacle, with either of the two parts (Velcro or snap fit) being positioned on module 100 and the other positioned on the distal end of the tether. The proximal tether ends 103p are arranged to be adhered to the patient's skin by adhesive material 105. Material 105 may be continuous strip or may be multiple strips to bind both tethers to the skin. While two tethers are shown, more than two tethers may also be present.

FIG. 1 illustrates one example of an arrangement to hold catheter 104 in place in bore 101 after catheter 104 has been inserted into bore 101. The proximal end 100p of module 100 has a circular compression fitting 102 adjacent to and surrounding bore 101 opening on proximal end 100p. The compression fitting 102 may be clamped and constricted around catheter 104 after its insertion in bore 101 so that catheter 104 is held in place. The compression fitting 102 may have one or more screw adaptations or wedge and ladder adaptations which can be tightened to constrict the fitting around the surface of catheter 104 to hold catheter 104 in place. Alternatively, compression fitting 102 may be absent and the inside surface of bore 101 coated with an adhesive that binds surfaces together. The adhesive may be configured to enable easy insertion of catheter 104 into bore 101. Once in place, adhesive contact with inserted catheter 104 enables a firm bind or hold of catheter 104 in module 100. Adhesives delivering such properties are known in the art. An example includes but is not limited to microencapsulated adhesive in a non-tack coating. Frictional and/or pressure contact of the microcapsules enables delivery of the adhesive onto the surfaces to be adhered together.

To manipulate module 100 and catheter 104 for use with a patient, catheter 104 is removed from its sterile packaging and its proximal end is inserted into an orifice or incision in the patient. The catheter distal end which extends externally from the patient is inserted into bore 101. The distal end of catheter 104 is pushed through bore 101 to provide an appropriate position of module 100 on catheter 104 portion extending externally from the patient. Clamp 102 or bore adhesive is activated to provide a firm hold of catheter 104 in bore 101. Alternatively, module 100 may be installed onto catheter 104 prior to catheter insertion into the patient. The installation is accomplished by placing the distal end of catheter 104 into bore 101 and positioning module 100 on catheter 104 at a location to provide appropriate proximal catheter 104 length for insertion into the patient. The proximal end of catheter 104 is then inserted into the patient. Appropriate handling of the sterile catheter during this procedure will assure sterility of the inserted proximal portion of catheter 104. Tethers 103 may be attached to module 100 by snap fits, Velcro® connection or adhesive, or tethers 103 may already be attached to module 100. Tethers 103 may be cut at proximal ends 103p to provide appropriate lengths of tethers 103 fitting the patient. Adhesive material 105 is applied to the tethers in place on the patient's skin to adhere tethers 103 to the skin.

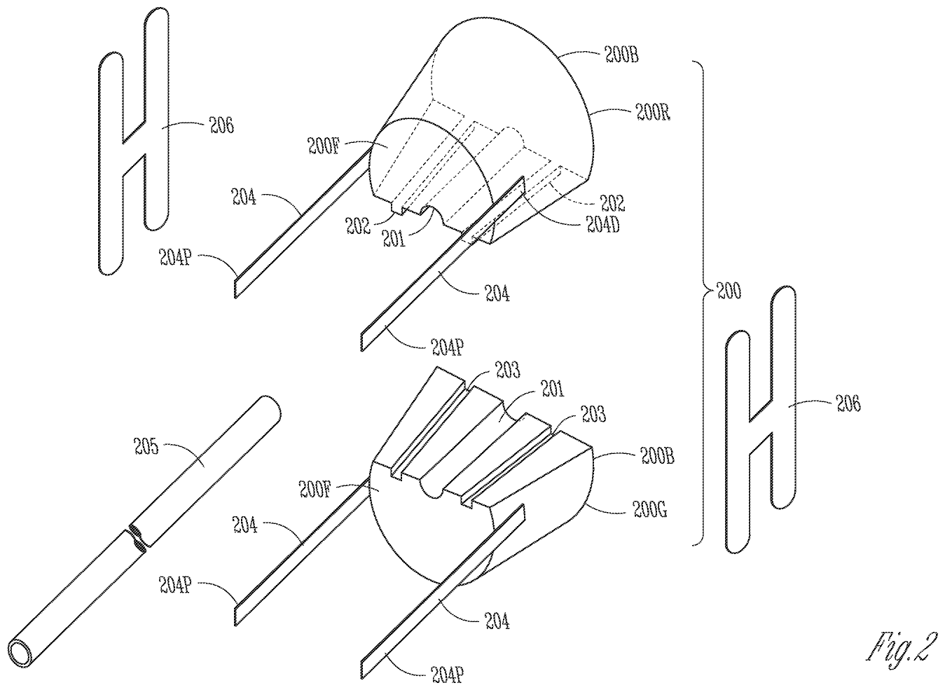

Embodiments of the section module and tethers according to the invention (FIG. 2) have dimensions suitable for use with intravenous catheters inserted in the arm or leg, Foley or vaginal catheters, any form of catheters for insertion into living organisms such as but not limited to humans, cannulas functioning as catheters to be inserted into living organisms and cannulas/catheters for connection to an indwelling portal functioning as a reservoir for a pharmaceutical agent. An example of such a reservoir is an indwelling insulin reservoir and pump. The overall dimension is configured so as to provide a comfortable, non-bulky arrangement on the patient's body.

FIG. 2 illustrates an embodiment directed to the sectioned module 200 having top module half 200r containing ridges 202 and bottom module half 200g containing grooves 203 (grooves and ridges may also be reversed with the top half having grooves and the bottom half having ridges). Module halves 200r and 200g divide along a plane including the center axis and running from front 200f to back 200b. The inner surfaces of the halves correspond to the midsection plane through the center axis of module 200. Halves 200r and 200g have mating ridges 202 and grooves 203 respectively on their inner surfaces. Ridges 202 and grooves 203 mate together in a binding fit when halves 200r and 200g are pressed together. Alternatively, instead of ridges 202 and grooves 203, the inner surfaces of halves 200r and 200g may be flat. The flat inner surfaces may contain an adhesive coating that will bind together the sections upon their mutual contact. The adhesive coating can be covered and protected by a release film or sheet to prevent inadvertent adhesive contact. Removal of the release film and mating the section halves 200r and 200g together will bind firmly the sections together. In another alternative (not shown) involving the flat inner surfaces, one or more Velcro® strips may be attached to the outer sides of halves 200r and 200g. Mating together halves 200r and 200g with flat inner surfaces and binding together the Velcro® strips provides a binding fit of the halves. Alternatively, snap fit projections may be provided in the outer sides of one of the halves and corresponding snap fit receptacles provided on the outer sides of the other half. Mating together halves 200r and 200g with snap fits and snapping together the snap fits provides a binding fit of the halves. Any combination of adhesive coating, Velcro® strips and/or snap fittings may also be employed. Also any combination of adhesive coating, Velcro® strips, snap fittings and/or ridges 202 and grooves 203 may also be present.

The inner surfaces of halves 200r and 200g whether ridged or flat are adapted and configured to provide the wall of the bore. The inner surfaces of both halves have concave channels 201 corresponding to part of the wall of the bore. When halves 200r and 200g are mated together as described above, channels 201 form the entire wall of the bore.

Channels 201 are configured to provide a binding fit of catheter 205 when halves 200r and 200g are mated with catheter 205 so that catheter 205 fits in the bore formed by channels 201. To provide the binding fit, channels 201 may be configured to have repeating ridges transverse to the long axis of bore 201 or the channels 201 may be flat and may have an adhesive coating on their surfaces. The adhesive coating may be covered with a release sheet or film to prevent inadvertent adhesive binding and/or contact as described above for the flat inner surfaces 200r and 200g. In this embodiment, the adhesive coating preferably would cover the inner surfaces 200r and 200g as well as channels 201 and would include the release sheet or film. Alternatively, both the ridges and the adhesive coating may be present. The ridges and/or adhesive coating of channels 201 are adapted to retain tightly and provide a binding fit of catheter 205 in the bore formed by channels 201. In an alternative embodiment (not shown), the concave channel 201 of one of halves 200r and 200g may corresponds to substantially or to essentially all of the wall of the bore and the other half of 200r and 200g may have a convex ridge in place of channel 201. The convex ridge mates with the concave channel. When mated together with catheter 205, the convex ridge presses against the catheter wall in channel 201 and provides a snug binding fit of catheter 205. The ridge and channel may optionally be coating with an adhesive and release sheet as described above.

Tethers 204 are attached to module halves 200r and 200g at their distal ends 204d by a binding adhesive, a Velcro® connection or by a snap fit arrangement of two parts (not shown), a snap protrusion and a snap receptacle, with either of the two parts of the Velcro® connection or snap fit being positioned on module 200r and 200g and the other positioned on the distal end of a tether. The proximal ends 204p may be cut to appropriate length and arranged to be adhered to the patient's skin by adhesive material 206. Material 206 may be continuous strip or multiple strips to bind all tethers to the skin. While two tethers are shown, three tethers, four tethers or more than four tethers may be present.

To manipulate module 200 and catheter 205 for insertion into a patient, catheter 205 is removed from its sterile packaging and its proximal end is inserted into the patient. Following catheter 205 insertion, its distal length which is external to the patient is placed into one of channels 201 of module 200. Halves 200r and 200g are mated together by snapping ridges 202 into grooves 203 or by mating adhesive coated flat inner surfaces together and thereby binding an appropriate section of catheter 205 into the bore formed by mating channels 201 together. Alternatively, the distal end of catheter 205 may be placed into one of the channels 201, halves 200r and 200g mated and bound together by snapping ridges and grooves or by adhesive to install catheter 205 in module 200. The proximal end of catheter 205 may then inserted into the patient. Appropriate handling of the sterile catheter during this procedure will assure sterility of the inserted proximal portion of catheter 205. Tethers 204 may be attached to module 200 by snap fits, Velcro® connection or adhesive or tethers 204 may already be attached to module 200. Tethers 204 may be cut at proximal ends 204p to provide appropriate lengths of tethers 204 fitting the patient. Adhesive material 206 is applied to the tethers in place on the patient's skin to adhere tethers 204 to the skin.

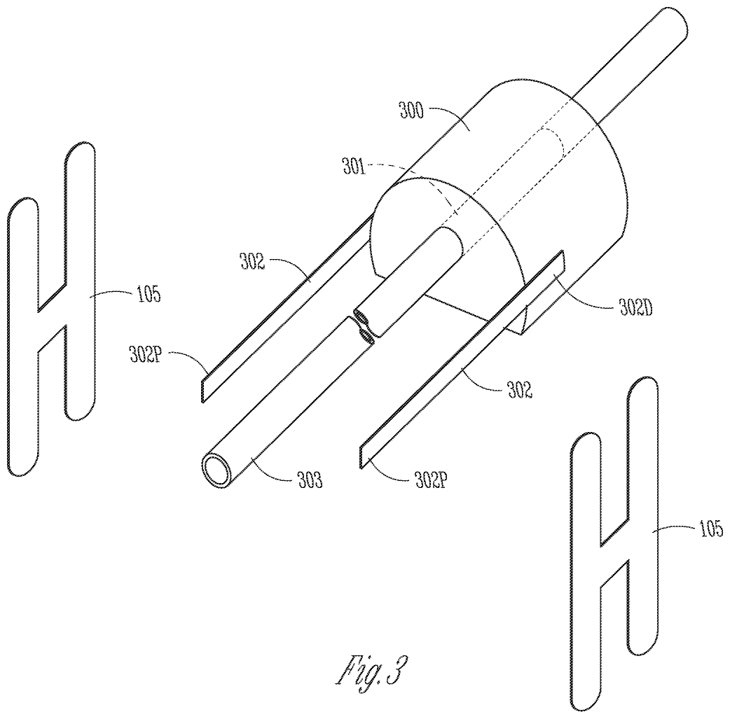

Embodiments of the conjoined module, catheter and tethers according to the invention (FIG. 3) have dimensions suitable for use with intravenous catheters inserted in the arm or leg, Foley or vaginal catheters, any form of catheters for insertion into living organisms such as but not limited to humans and cannulas functioning as catheters to be inserted into living organisms and cannulas/catheters for connection to an indwelling portal functioning as a reservoir for a pharmaceutical agent. An example of such a reservoir is an indwelling insulin reservoir and pump. The overall dimension is small so as to provide a comfortable, non-bulky arrangement on the patient's body.

FIG. 3 illustrates an embodiment directed to a permanently conjoined device comprised of catheter 303 and module 300. Module 300 is a unitary body having bore 301. Bore 301 may be coated with adhesive as described above for FIG. 1. At manufacture, catheter 303 is inserted into bore 301 and adhesive on the bore wall is set to bind catheter 303 to the bore wall of module 300. The preformed device may be sterilized and placed in a sterile package for later use.

Tethers 302 may be provided preferably as separate components to be attached to module 300 upon use. Tethers 302 may be attached to module 300 at their distal ends 302d by a binding adhesive, or by a Velcro connection or a snap fit arrangement of two parts (not shown), a snap projection and snap receptacle, with either of the two parts (Velcro® connection and snap fit) being positioned on module 300 and the other positioned on the distal end of the tether. The proximal ends 302p are arranged to be adhered to the patient's skin by adhesive material 304. Material 304 may be continuous strip or multiple strips to bind both tethers to the skin. While two tethers are shown, more than two tethers may also be present.

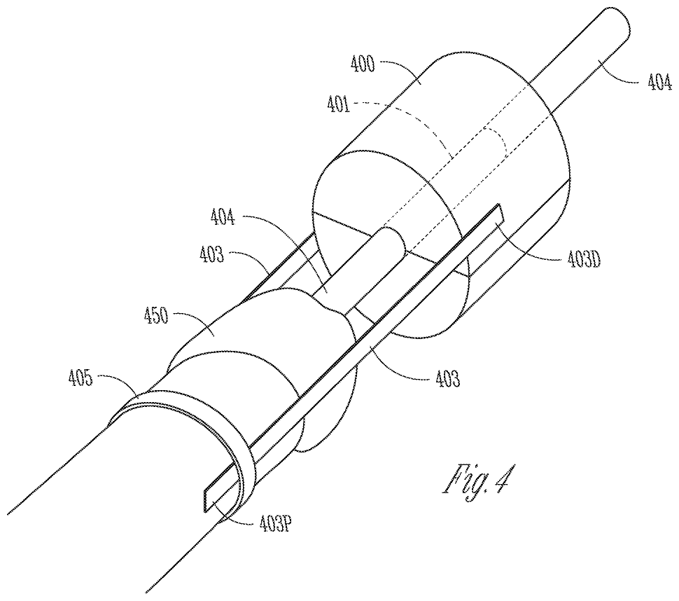

Embodiments of the module and tethers according to the invention (FIG. 4) have dimensions suitable for use with a Foley catheter to be inserted into the urethra of a male organ. The overall dimension is configured so as to provide a comfortable, non-bulky arrangement on the patient's body.

FIG. 4 illustrates the device with a Foley catheter inserted into a male organ 450. An external portion of Foley catheter 404 extends through the bore 401 of module 400. Tethers 403 have distal ends 403d attached to the external surface of module 400 and proximal ends 403p contacting the skin of male organ 450. Adhesive material 405 binds proximal ends 403p to the skin of male organ 450. Preferably, the Foley catheter is first inserted into the patient and the unitary body module of FIG. 1 or the section module of FIG. 2 is subsequently installed onto the external portion of the Foley catheter. Preferably, the section module of FIG. 2 is employed with the Foley catheter and the binding of the module and Foley catheter as well as binding of the section halves of the module of FIG. 2 are accomplished by adhesive coating. Before installation, the flat inner surfaces of the module halves 200r and 200g and channels 201 have adhesive coatings protected with release film or strip. The release film is removed, module 200 section halves mated together and simultaneously the distal portion of the Foley catheter is fitted into the bore formed by channels 201. Pre-attached tethers 204 are cut to appropriate length and attached to the skin of the male organ 450 with adhesive material. The result is the Foley catheter with module 400 depicted in FIG. 4.

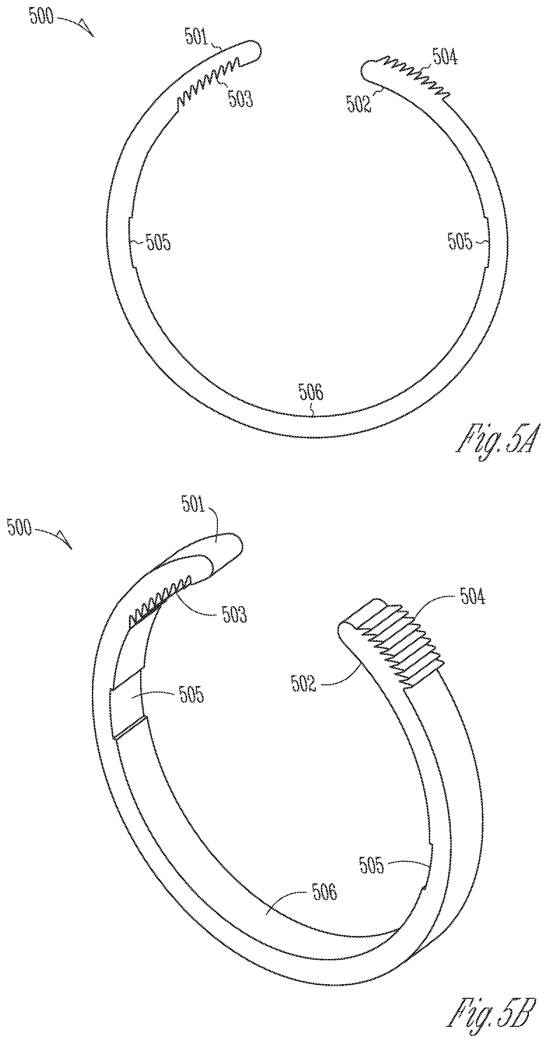

FIGS. 5A and 5B illustrate a split ring alternative for use with a Foley catheter and module. The split ring substitutes for an adhesive material for holding the tethers to the male member. Split ring 500 is flexible. Applying force at part 501 moves part 501 over part 502 so as to provide a smaller diameter of the flexible ring. Teeth 503 and teeth receivers groves 504 of parts 501 and 502 constitute the snap fit and hold the closed ring in place. The inner surface of ring 500 may be coated with an adhesive or tacky substance to provide increased adherence of the ring 500 to a male member. Multiple slots 505 (two shown, more may be present) are located on the inner surface of the ring and are positioned for insertion of tethers. The slots may include adhesive material to enable affixing the tethers.

For all embodiments of the devices according to the invention including but not limited to the illustrations of FIGS. 1, 2, 3 and 4, the size of bore 101, size of the bore formed by grooves 201 or the size of bore 301 and 401 may be adapted to appropriately fit the outer diameter of catheter 104, 205, 303 or 404. The outer diameter of a catheter varies as a function of its use, such as but not limited to intravenous catheter for short length, short duration insertion into an arm or leg vein, long length long duration insertion into an arm or leg vein or long term insertion into a urethra of a male or female patient. Accordingly, the modules may be designed and constructed to fit the variability of catheter size.

The configurations of module 100, 200, 300 and 400 may be appropriate to provide a comfortable. non-binding arrangement under clothing, hospital garments and/or hospital surgery and recovery rooms and/or hospital patient rooms. While the configurations of modules 100, 200, 300 and 400 may include edges and corners, rounded edges and corners and preferably rounded exterior shapes will provide appropriate comfort in this regard. Preferably, the size of modules 100, 200, 300 and 400 may be small, such as but not limited to approximately 2 to 30 centimeters in length and 2 to 20 centimeters in width and height, more preferably up to 15 to 20 centimeters in length, width and height. For substantially rounded cylinder shaped modules, the size preferably may be, but is not limited to, approximately up to 30 centimeters in length and approximately up to 20 centimeters in diameter. Preferably for semi-cylinder-shaped modules, the sizes may range from about 2 to 20 centimeters, more preferably up to about 10 to 15 centimeters in all directions.

Tethers may be of appropriate lengths and widths to provide comfortable fit for the patient. Tether sizes may range up to 50 to 70 centimeters in length and up to 1 to 5 centimeters in width. The tether lengths may be cut to provide a custom fit for each patient. The width of the tethers in part contributes to their rigidity. The tethers have an appropriate rigidity to prevent accidental movement of the catheter relative to its placement in the patient.

Embodiments of devices according to the invention including modules 100, 200, 300 and 400, tethers 103, 204, 302 and 403 and ring 500 may be constructed of any appropriate material suitable for external medical use. The material for the modules and tethers may be substantially strong and substantially inflexible. This module and tether material may be an extrudable or moldable plastic such as a high temperature thermoplastic or thermoset polyolefin, polyester, polyurethane, polyurea, polycarbonate, polyamide, polyacrylic, polyethylene, polypropylene or any combination thereof. The module and tethers may alternatively be metal, any kind of rubber, shape retaining silicone, or a shape retaining gel. The choice of material for the modules will be appropriately made so that it will be compatible with the polymer material of the catheter. The material of the module is selected so that it will not cause degradation of the catheter material and will enable adhesive binding between these materials and with a patient's skin. The material of ring 500 is selected so that the ring is strong and flexible.

Embodiments of tethers 103, 204, 302 and 403 are not required to be the same material as the modules but may also preferably be made of similar material as the module. The tether material may appropriately deliver strength and some flexibility combined with stiffness at the same time. The stiffness/flexibility quotient should be balanced so that movement of the conjoined catheter and module relative to placement on and in a patient will not occur.

Embodiments of the device according to the invention, and especially the catheter component may be fitted with a micro-pressure sensor to detect and signal rotational, torsional and/or translational force applied to the device with catheter inserted in a patient. The sensor can be connected to appropriate wireless transmission devices at the patient bedside. The transmission devices can be adapted to detect the pressure signal from the sensor and transmit a message to the patient's nursing station. In this manner, a patient's discomfort and attempt to move the catheter may be handled in an appropriate, non-emergency manner. Such a micro-pressure sensor device is, for example described in Hasenkamp et. al, Biomed Microdevices, (2012) 14:819-828.

Embodiments according to the invention directed to incorporation of the adhesive material include but are not limited to band-aids, adhesive bandages, butterfly bandages, adhesive strips, constructed adhesive material and similar compositions having tacky adhesive coatings on woven or nonwoven textile material. The adhesive material may be constructed with snap fittings as described above for connection with the proximal ends of the tethers, especially when the arrangement of the device does not necessitate cutting the tethers to size. The adhesive material may alternatively be constructed with pockets into which will fit the proximal ends of the tethers. the pockets may be internally coated with adhesive substance to firmly hold the proximal ends of the tethers. This alternative preferably may be applicable when the tethers are to be cut to size. With ring alternative 500, the ring may include slots 505 into which the sized tethers may be inserted. The slots may include an adhesive substance to firmly hold the proximal ends of the tethers in slots 505

A preferred adaptation of the securing device according to the invention is its use in conjunction with a Foley catheter as shown by FIG. 4. Preferably, a section module such as but not limited to the section module of FIG. 2 with multiple tethers may be appropriately employed with a Foley catheter as shown by FIG. 4. While FIG. 4 illustrates use with a male patient, the Foley catheter/securing device arrangement may be applied as well to a female patient. The adhesive holding the tethers in place would be attached to the female patient's lower abdomen, trunk and/or one or both thighs.

According to the invention, embodiments directed to the Device for securing a catheter may be packaged as a kit including as separate items, the module, one or more tethers, adhesive material and adhesive and appropriate tools for assembly if needed. A separate but associated package to be included with the kit may be the appropriately sized and designed catheter suitable for the purpose intended. The catheter package may be provided as a sterilized item while the Device package need not be sterile.

### SUMMARY STATEMENTS

The inventions, examples and results described and claimed herein may have attributes and embodiments include, but not limited to, those set forth or described or referenced in this application.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, text book or other referenced material or document. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

The written description of this patent application includes all claims. All claims including all original claims are hereby incorporated by reference in their entirety into the written description portion of the specification and the right is reserved to physically incorporated into the written description or any other portion of the application any and all such claims. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims.

1. A device for securing a catheter, comprising a module with a front, a back and a bore traversing the module from front to back with the bore configured to fit around the catheter, and at least two tethers having proximal and distal ends, the distal ends being removably attached to the module.

2. The device of claim 1, further comprising an adherent material for removable attachment to the proximal ends of the tethers.

3. The device of claim 1, wherein the module has an outer surface, a midportion, front and back ends and the bore traversing the module from the front end to the back end and through the midportion.

4. (canceled)

5. The device of claim 1, wherein the bore of the module has a wall adapted to provide a binding fit around the catheter and the binding fit will substantially prevent movement of the catheter relative to the wall.

6. (canceled)

7. (canceled)

8. The device of claim 1, wherein the module is constructed of a thermoplastic or thermoset organic polymer, a cross linked organic polymer, a latex, a natural or synthetic rubber, a silicone polymer, a shape retaining gel or a metal.

9. The device of claim 1, wherein the tethers are rigid or semi-flexible rods, slats, planks or strips and are permanently or removably attached to the module.

10. (canceled)

11. (canceled)

12. (canceled)

13. The device claim 1, wherein the module is divided into at least two sections and tethers are permanently or removably attached to each module section.

14. (canceled)

15. (canceled)

16. The device of claim 2, wherein the adherent material is a flexible adhesive material adapted to adhere to skin.

17. (canceled)

18. The device of claim 2, wherein the adherent material is adapted to have one or more pockets into which fit the proximal ends of the tethers.

19. (canceled)

20. The device of claim 2, wherein the adherent material and tethers are adapted to have snap fasteners for connecting the proximal ends of the tethers to the adherent material.

21. The device of claim 1 adapted to be used with a Foley catheter.

22. The device of claim 1 adapted to be used with a venous or arterial catheter.

23. A method for securing a catheter positioned or to be positioned in a vein, artery or duct of a patient through use of a device of claim 1, the catheter having a portion external and a portion internal to the patient, comprising installing the external portion of the catheter within the bore of the device, and the applying the adherent material to the skin of the patient to adhere the tethers to the skin.

24. The method of claim 23, wherein the catheter is inserted in the patient before installation of the device on the catheter.

25. The method of claim 23, wherein the device is installed onto the catheter before the catheter is inserted into the patient.

26. The method of claim 23, wherein the tethers connected with the adherent material, the fit of the external portion of the catheter within the bore of the module, and the adherence of the adherent material to the skin of the patient cooperate to resist movement of the catheter positioned within the vein, artery or duct of the patient.

27. The method of claim 23, wherein the catheter cannot be moved or removed from its position within the vein, artery or duct without un-adhering the adherent material from the skin of the patient and disengaging the module from the external portion of the catheter.

28. The method of claim 23, wherein the catheter is a Foley catheter, the Foley catheter is inserted into the urethra of a male patient and the skin is the skin of a penis of a patient.

29. The method of claim 23, wherein the catheter is an intra venous catheter, the intravenous catheter is inserted into the vein of an arm or leg of a patient and the skin is the skin of the arm or leg.

30. (canceled)

31. A device for securing a catheter, comprising a module adapted to fit around the catheter, tethers having proximal and distal ends, the distal ends being removably attached to the module, and an optional adherent material to which can be removably attached the proximal ends of the tethers, wherein
the module has polygon shape, the dimensions of which enable unencumbered use with a catheter, the module has an outer surface, a midportion, two ends and a bore traversing the module from one end to the other through the midportion, the bore having a wall that is configured to provide a mechanical and/or adhesive binding fit around the catheter;
the module is a unitary construct or is a sectional construct;
as a sectional construct, the module is divided in at least two sections along a plane that intersects the bore, the sections having outer and inner surfaces, the outer surface of each section being the outer surface of the module, the inner surface of each section having a concave portion that forms at least a part of the wall of the bore, the inner surfaces of the sections adapted to be removably and adherently and/or mechanically secured together;
the tethers are rigid or semi-flexible, are permanently or removably attached to the module and have a rod-like or slat-like shape;
the optional adherent material is a flexible adhesive material adapted to removably adhere to skin and to be removably affixed to the proximal ends of the tethers;
the affixation of the tethers to the optional adhesive material is provided by adhesive binding of the adhesive material to the proximal ends of the tethers, or by pockets in the adhesive material into which fit the proximal ends of the tethers; and
the module and tethers are made of thermoplastic or thermoset organic polymer, metal, latex, natural or synthetic rubber, cross linked organic polymer or silicone and the optional flexible adhesive material is made of a non-woven or cast cellulosic or polymeric material coated with dry tacky adhesive over which is laid a removable backing.

32. (canceled)

33. (canceled)

34. (canceled)

35. (canceled)

36. (canceled)

37. (canceled)

38. (canceled)
